SEQUENCE LISTING (7 sequences)

SEQ ID NO: 1 - DNA, 22 bp, Mus musculus
aacaactgggctttgcacattg

SEQ ID NO: 2 - DNA, 21 bp, Mus musculus
gtttatcagggtttcttctag

SEQ ID NO: 3 - DNA, 36 bp, Artificial sequence
  Synthetic PCR primer
tttaaaggccggccatgccagagccagcgaagtctg

SEQ ID NO: 4 - DNA, 36 bp, Artificial sequence
  Synthetic PCR primer
tttaaaggccggccttacttgtacagctcgtccatg

SEQ ID NO: 5 - DNA, 25 bp, Mus musculus
cccaaagtcgctctgagttgttatc

SEQ ID NO: 6 - DNA, 24 bp, Mus musculus
gaagaagcgggagaaatggatatg

SEQ ID NO: 7 - DNA, 23 bp, Artificial sequence
  Syntehtic PCR primer
ccaggcgggccatttaccgtaag

(19) United States
(12) Patent Application Publication
(10) Pub. No.: US 2013/0074200 A2
(43) Pub. Date: Mar. 21, 2013

(54) INBRED C57BL/6 ES CELLS WITH HIGH DEVELOPMENTAL CAPACITY

(71) Applicant: Sang Kim, Greenlawn, NY (US)

(72) Inventor: Sang Kim, Greenlawn, NY (US)

(73) Assignee: Cold Spring Harbor Laboratory, Cold Spring Harbor, NY (US)

(21) Appl. No.: 13/011,799

(22) Filed: Jan. 21, 2011

(51) Int. Cl. A01K 67/027; C12N 5/10; C12N 5/07

(52) U.S. Cl. 800/18; 435/354

(57) ABSTRACT

Described herein are inbred B6 ES cell lines that exhibit high developmental capacities and have a number of advantages over ES cell lines already available. First, they can be used for gene targeting and have a high percentage of germline transmission when injected into diploid host blastocysts (˜50-80%). Second, these ES cell lines can successfully be used to generate live pups by tetraploid blastocyst complementation, producing a high percentage (15-20%) of mice that are entirely inbred B6 ES cell derived. Third, these ES cells lines can be used to rapidly generate mice that are homozygous for a gene of interest. These advantages indicate that the inbred B6 ES cells provided here facilitate the rapid generation of inbred B6 mouse models in a cost-effective and efficient manner.

### GOVERNMENT SUPPORT

This invention was made with U.S. Government support under grant NCI 10/35601, awarded by the National Cancer Institute. The U.S. Government has certain rights in this invention.

### BACKGROUND

Mouse embryonic stem (ES) cells are pluripotent and can contribute to all tissues of a mouse after blastocyst injection. They can also be genetically manipulated with relative ease and have been used for the generation of gene-targeted mice after introduction of targeted mutations into the genome (see e.g., Doetschman T et al. 1987). Most of the germline-competent mouse ES cell lines in use today are outbred lines derived from 129 mouse substrains Inbred 129 are also used, but typically exhibit a lower developmental capacity than outbred ES cell lines. The genetic background of 129-derived ES cell lines is often not ideal for biomedical science, such as, certain genetic studies of immunology, neurobiology and physiology. Further, 129 ES cell-derived chimeras are frequently difficult to breed and often show abnormalities in anatomy, physiology, and behavior. ES cell lines derived from other inbred mouse strains are available and, although the use of inbred ES cell lines is advantageous from an experimental point of view, inbred ES cell lines have been reported to exhibit a significantly decreased developmental capacity, as manifested in low contribution rates to tissues of chimeras, low or no contribution to the germline of chimeric mice, and very low efficiency or failure in sustaining development of live mice after tetraploid complementation (see, e.g., Eggan et al., PNAS, 2001 (May) 6209-6214; and Li et al., Reproduction, 2005 130:53-59). It would be useful to have an inbred mouse ES cell line with improved developmental capacity.

### SUMMARY

Described herein are inbred C57BL/6 ES cell lines of (that exhibit) high developmental capacity (e.g., developmental capacity sufficient to support the generation of live pups via tetraploid blastocyst complementation), such as inbred C57BL/6J ES cell lines of high developmental capacity. Also described herein are methods of deriving inbred C57BL/6 ES cell lines of high developmental capacity (e.g., inbred C57BL/6J ES cell lines of high developmental capacity), such as methods of deriving such inbred ES cell lines from frozen or cryopreserved embryos. Methods of using inbred C57BL/6 ES cell lines of high developmental capacity (e.g., inbred C57BL/6J ES cell lines of high developmental capacity) to produce mice, such as genetically modified inbred C57BL/6 mice, are also described. Also described are mice derived from inbred C57BL/6 ES cell lines of high developmental capacity (e.g., inbred C57BL/6J ES cell lines of high developmental capacity), such as germline-competent ES cell chimeras and ES cell derived mice obtained via tetraploid blastocyst complementation.

Inbred C57BL/6 ES cell lines of high developmental capacity (e.g., inbred C57BL/6 ES cell lines of high developmental capacity) described herein include, for example, ES cell line MK6, described herein—and inbred C57BL/6 ES cell lines, such as inbred C57BL/6J ES cell lines, that exhibit developmental capacity similar to that of ES cell line MK6. Inbred C57BL/6 ES cell lines of high developmental capacity also include, for example, ES cell line MK6V, described herein, such as inbred C57BL/6J ES cell lines, that exhibit developmental capacity similar to that of ES cell line MK6V. Such inbred ES cell lines exhibit developmental capacity sufficient to support the generation of live pups via tetraploid blastocyst complementation.

In some embodiments, inbred C57BL/6 ES cells provided herein are euploid. Alternatively, inbred C57BL/6 ES cells provided herein can be aneuploid. Typically, the inbred C57BL/6 ES cells of high developmental capacity described herein have the ability to grow in an undifferentiated culture for at least 1 year on feeder cells and/or in the presence of Leukemia Inhibitory Factor (LIF). Further, the inbred C57BL/6 ES cells of high developmental capacity described herein exhibit ES cell morphology on feeder cells.

Also disclosed are populations of inbred C57BL/6 mouse ES cells of high developmental capacity, such as populations of inbred mouse ES cells derived from or an ancestor of which was derived from the MK6 ES cell line or derived from the MK6V ES cell line. Further provided are populations of inbred C57BL/6 mouse ES cells that are substantially identical to mouse ES cells of the MK6 ES cell line or substantially identical to mouse ES cells of the MK6V ES cell line described herein.

In some embodiments, an inbred C57BL/6 ES cell population is provided that can be used to generate live pups via tetraploid embryo complementation. In some embodiments, an inbred C57BL/6 ES cell population is provided that contributes to the germline of pups generated via tetraploid embryo complementation. In some embodiments, the ES cell population comprises at least one cell that comprises a genetic modification. In some embodiments, the genotype of essentially all cells in the population is substantially the same (e.g., all cells exhibit the same genotype including a specific genetic modification). In some embodiments, the genetic modification is a heterologous nucleic acid construct stably integrated into the genome of the cell. In some embodiments, the genetic modification is a knockout (e.g., a knockout of a gene of interest), a knock-in (e.g., a knock-in of a construct of interest), a viral vector (e.g., a retroviral vector comprising a heterologous expression cassette), or a randomly integrated nucleic acid construct.

Methods for genetically modifying an ES cell line are well known to those of skill in the art, and some exemplary, non-limiting methods are described in Notarianni and Evans, Embryonic stem cells: a practical approach, Oxford University Press, 2006, ISBN 0198550006; Joyner, Gene targeting: a practical approach, Oxford University Press, 2000, ISBN 019963792X; Jackson and Abbott, Mouse Genetics and Transgenics: A Practical Approach, Oxford University Press, 2000, ISBN 0199637083; Kmiec, Gene Targeting Protocols (Methods in Molecular Biology), Humana Press, 2000, ISBN 0896033600; Tymms and Kola, Gene Knockout Protocols (Methods in Molecular Biology), Humana Press, 2001, ISBN 0896035727; the teachings of all of which as related to genetic modification of mouse embryonic stem cells are incorporated herein by reference.

For example, in some embodiments, the genetic modification is a knock-in of an expression construct into the Rosa26 locus. In some embodiments, the expression construct is a reporter construct, for example, a Histone 2B/GFP fusion protein (H2B-GFP). Other useful loci and expression or reporter constructs will be readily apparent to those of skill in the art and the invention is not limited in this respect.

Also provided is a pre-implantation embryo comprising a (at least one, one or more) inbred C57BL/6 mouse ES cell of high developmental capacity, such as an ES cell of an inbred C57BL/6 ES cell line of high developmental capacity as provided herein, or an inbred ES cell derived from such an inbred ES cell line. In some embodiments, the pre-implantation embryo is a blastocyst. In some embodiments, the pre-implantation embryo comprises a tetraploid cell population (e.g., a tetraploid trophoblast cell population). In some embodiments, the preimplantation embryo includes a tetraploid cell population, such as a tetraploid cell population derived from the fusion product of the two blastomeres of a diploid two cell stage embryo, and a heterologous, diploid, inbred C57BL/6 ES cell population.

Another embodiment is a mouse that comprises a cell derived from an inbred C57BL/6 ES cell of high developmental capacity as provided herein, such as a cell derived from an MK6 ES cell or an MK6V ES cell or a derivative of either. One embodiment is a mouse whose germline comprises a cell derived from an inbred C57BL/6 ES cell of high developmental capacity as provided herein, such as from an MK6 ES cell or an MK6V ES cell or a derivative of either. A further embodiment is a mouse that consists essentially of cells derived from an inbred C57BL/6 ES cell (e.g., from an inbred C57BL/6J ES cell) of high developmental capacity as provided herein, such as a mouse that consists essentially of cells derived from an MK6 ES cell or a mouse that consists essentially of cells derived from an MK6V ES cell or a derivative of either. In one embodiment, the mouse is a mouse derived from an inbred C57BL/6 ES cell of high developmental capacity or from a derivative thereof, as described herein, via tetraploid embryo complementation, such as a mouse derived from MK6 ES cells or MK6V ES cells, or a derivative of either, via tetraploid embryo complementation.

Also described are cells that are derivatives of an inbred C57BL/6 ES cell of high developmental capacity as provided herein, such as cells that are derivatives of an MK6 ES cell or cells that are derivatives of an MK6V ES cell. In some embodiments, the derivative cell is a pluripotent daughter cell of an inbred C57BL/6 ES cell of high developmental capacity as provided herein, such as a pluripotent daughter cell of an MK6 ES cell or a pluripotent daughter cell of an MK6V ES cell. In some embodiments, the derivative cell is a genetically modified daughter cell of an inbred C57BL/6 ES cell (e.g., of an inbred C57BL/6J ES cell) of high developmental capacity as provided herein, such as a genetically modified daughter cell of an MK6 ES cell or a genetically modified daughter cell of an MK6V ES cell. In some embodiments, the derivative cell is a differentiated daughter cell of an inbred C57BL/6 ES cell provided herein, such as a differentiated daughter cell of an MK6 cell or a differentiated daughter cell of an MK6V ES cell. In some embodiments, the derivative cell is a differentiated genetically modified daughter cell of an inbred C57BL/6 ES cell of high developmental capacity as provided herein, such as a differentiated genetically modified daughter cell of an MK6 cell or a differentiated genetically modified daughter cell of an MK6V ES cell.

Also provided are inbred C57BL/6 mouse ES cells derived from a frozen inbred C57BL/6 embryo or inbred C57BL/6 mouse ES cells derived from a cryopreserved, inbred C57BL/6 embryo. In some embodiments, the frozen inbred C57BL/6 embryo or cryopreserved inbred C57BL/6 embryo is a C57BL/6J embryo and the inbred C57BL/6 mouse ES cells of high developmental capacity are inbred C57BL/6J ES cells. In some embodiments, pluripotent cells, for example, genetically modified pluripotent ES cells derived from an inbred C57BL/6 mouse ES cell, cell population, or cell line derived from a frozen or cryopreserved, inbred C57BL/6 embryo are provided. For example, in some embodiments, genetically modified ES cells derived from an inbred C57BL/6J mouse ES cell, cell population, or cell line derived from a frozen or cryopreserved, inbred C57BL/6J embryo are provided. In some embodiments, the inbred C57BL/6 ES cell, cell population, cell line, or pluripotent derivative of high developmental capacity generates live pups after tetraploid blastocyst complementation, for example, more than about 5%, more than about 10%, more than about 15%, or more than about 20% live pups (as measured by live pups at term in relation to tetraploid complemented embryos transferred) after tetraploid blastocyst complementation. In some embodiments, the inbred C57BL/6 ES cell, cell population, cell line, or pluripotent derivative generates more than about 5%, more than about 10%, more than about 15%, or more than about 20% live pups at term (as measured by live pups at term in relation to tetraploid complemented embryos transferred) after complementation of tetraploid cBrd/cBrd/cr or BALB/c blastocysts.

In some embodiments, a mouse is provided that comprises a (at least one, one or more) cell derived from an inbred C57BL/6 ES cell line, ES cell, ES cell population of high developmental capacity, or a pluripotent derivative thereof, as provided herein. In some embodiments, the mouse is generated by injection of a (at least one, one or more) pluripotent cell derived from an ES cell or cell line provided herein, for example, of a genetically modified pluripotent cell derived from an ES cell line provided herein (e.g. MK6 or MK6V), into a host blastocyst. In some embodiments, the blastocyst is a diploid blastocyst. In some embodiments, the blastocyst is a tetraploid blastocyst. In some embodiments, essentially all cells of the mouse are derived from the injected pluripotent cell, for example, from the injected MK6 ES cell, the injected MK6V ES or a derivative of either, such as a genetically modified derivative of either. In some embodiments, essentially all cells of the mouse are derived from an injected pluripotent cell, for example, from an injected inbred C57BL/6 ES cell derived from a frozen inbred C57BL/6 embryo or from a cryopreserved inbred C57BL/6 embryo as described herein. In some embodiments, at least one germ cell of the mouse is derived from the injected pluripotent cell. In some embodiments, essentially all germ cells of the mouse are derived from the injected pluripotent cell.

Also described is a method of deriving a mouse ES cell line from a frozen inbred C57BL/6 embryo or cryopreserved inbred C57BL/6 embryo. In some embodiments, the method comprises providing a frozen inbred C57BL/6 mouse embryo or a cryopreserved inbred C57BL/6 mouse embryo; culturing the embryo under appropriate conditions for production of/development to the blastocyst stage, thereby producing a blastocyst; incubating the blastocyst on feeder cells for a time sufficient and under conditions appropriate to form an inner cell mass (ICM) outgrowth; disaggregating the inner cell mass outgrowth to produce an ES cell population; and subculturing an ES cell population derived from the ICM outgrowth to establish an ES cell line. Appropriate conditions, in some embodiments, include incubation at a temperature of about 30-39° C., for example, of about 37° C. Such conditions will result in thawing of the embryo. In some embodiments, the method comprises thawing the embryo, for example, by incubating the embryo at a temperature above freezing, for example, in a water bath, for a time sufficient for the embryo to thaw. In some embodiments, the embryo has been frozen. In some embodiments, the embryo has been cryopreserved. In some embodiments, the embryo has been frozen or cryopreserved at the eight-cell stage. In some embodiments, the embryo has been frozen or cryopreserved for less than three months, for at least 3 months, for at least 6 months, for at least 1 year, for at least 2 years, or for at least 3 years.

Some aspects of this invention provide an inbred mouse C57BL/6 embryonic stem (ES) cell that exhibits high developmental capacity. In some embodiments, the ES cell line is a C57BL/6J ES cell line. In some embodiments, the ES cell exhibits substantially the same developmental capacity as inbred ES cell line MK6 or substantially the same developmental capacity as inbred ES cell line MK6V. In some embodiments, the ES cell is derived from the MK6 ES cell line or the MK6V ES cell line. Some aspects of this invention provide a population of inbred C57BL/6 mouse ES cells, comprising one or more of the ES cells described herein. In some embodiments, the ES cell population contributes to the germline in pups generated after tetraploid embryo complementation. In some embodiments, the ES cell population comprises at least one cell that comprises a genetic modification. In some embodiments, essentially all cells in the population comprise the same genetic modification. In some embodiments, the genetic modification is a heterologous nucleic acid construct stably integrated into the genome of the cell. In some embodiments, the genetic modification is a knockout, a knock-in, a viral vector, or a randomly integrated nucleic acid construct. In some embodiments, a pre-implantation embryo is provided that comprises any of the inbred mouse C57BL/6 ES cells or populations of ES cells described herein. In some embodiments, the pre-implantation embryo is a blastocyst. In some embodiments, the embryo comprises a tetraploid trophoblast cell population. In some embodiments, the cell population of the embryo consists of an inbred C57BL/6 ES cell population described herein and a tetraploid cell population. In some embodiments, a mouse is provided that comprises a cell derived from any of the inbred C57BL/6 ES cells described herein or from any inbred C57BL/6 ES cell population described herein. In some embodiments, a mouse is provided that consists essentially of cells derived from any of the inbred C57BL/6 ES cells described herein or from any inbred C57BL/6 ES cell population described herein. In some embodiments, a differentiated cell is provided that is derived from any of the inbred C57BL/6 ES cells described herein or from any inbred C57BL/6 ES cell population described herein.

In some embodiments, mouse ES cells of high developmental capacity are provided, wherein the ES cells are derived from an inbred C57BL/6 embryo, and wherein the ES cells, or pluripotent cells derived from the ES cells or substantially identical to the ES cells, generate live pups after tetraploid blastocyst complementation. In some embodiments, the ES cells, or the pluripotent cells derived from the ES cells or substantially identical to the ES cells, generate more than 5%, more than 10%, more than 15%, more than 20%, or more than 25% live pups (live pups at term/tetraploid complemented embryos transferred) after tetraploid blastocyst complementation. In some embodiments, tetraploid cBrd/cBrd/cr or BALB/c blastocysts are used for the tetraploid blastocyst complementation. In some embodiments, the embryo is a C57BL/6J embryo. In some embodiments, the embryo has been frozen. In some embodiments, the embryo has been cryopreserved. In some embodiments, the embryo has been frozen or cryopreserved at the eight-cell stage. In some embodiments, the embryo has been frozen or cryopreserved for at least 3 months, at least 6 months, at least 1 year, at least 2 years, or at least 3 years. In some embodiments, the embryo comprises a genetic manipulation, for example, a knock-out, or a knock-in. In some embodiments, the ES cells exhibit a normal karyotype. In some embodiments, the cells exhibit a 40,XY karyotype. In some embodiments, the pups generated are male. In some embodiments, the cells in the population exhibit a 39,X0 karyotype. In some embodiments, the pups generated are female.

In some embodiments, a pluripotent cell derived from any of the inbred C57BL/6 ES cells described herein is provided. In some embodiments, a differentiated cell derived from any of the inbred C57BL/6 ES cells described herein is provided. In some embodiments, the pluripotent or differentiated cell derived from any of the inbred C57BL6 ES cells described herein is comprised in a mouse. In some embodiments, the ES cell, or the pluripotent or differentiated cell derived from the ES cell, comprises a genomic modification. In some embodiments, the ES cell, or the pluripotent or differentiated cell derived from the ES cell, comprises a heterologous nucleic acid construct stably inserted into its genome.

In some embodiments, a mouse is provided that comprises a cell derived from any of the inbred C57BL6 ES cells or ES cell populations described herein. In some embodiments, a mouse is provided that is generated by injection of a pluripotent cell derived from any of the inbred C57BL/6 ES cells or ES cell populations described herein into a blastocyst. In some embodiments, the blastocyst is a diploid blastocyst. In some embodiments, the blastocyst is a tetraploid blastocyst. In some embodiments, essentially all cells of the mouse are derived from the injected ES cell(s). In some embodiments, at least one germ cell of the mouse is derived from the injected ES cell.

In some embodiments, a method for deriving a mouse ES cell line from a frozen or cryopreserved embryo is provided that comprises (a) providing a frozen or cryopreserved, inbred C57BL/6 mouse embryo; (b) culturing the embryo until the blastocyst stage; (c) incubating the blastocyst on feeder cells for a time sufficient and under conditions appropriate to form an inner cell mass (ICM) outgrowth; (d) disaggregating the inner cell mass outgrowth; and (e) subculturing an ES cell population derived from the ICM outgrowth to establish an ES cell line. In some embodiments, the method further comprises thawing the embryo.

In some embodiments, (b) comprises culturing the embryo in KSOM-AA medium; and/or (c) comprises incubating the blastocyst on feeder cells in ES cell culture medium. In some embodiments, the embryo has been frozen or cryopreserved at the eight-cell stage. In some embodiments, the embryo has been frozen or cryopreserved for at least 3 months, at least 6 months, at least 1 year, at least 2 years, or at least 3 years. In some embodiments, the ES cell culture medium comprises (i) Knockout DMEM (Invitrogen/GIBCO); (ii) knockout serum replacement (KSR, Invitrogen/GIBCO); (iii) 1,000 units/ml leukemia inhibitory factor (Chimicon); (iv) 0.1 mM 2-mercaptophenol (Sigma); (v) 2 mM glutamax (Invitrogen); (vi) 1 mM sodium pyruvate; and/or (vii) 0.1 mM nonessential amino acids.

In some embodiments, an ES cell is provided, wherein the ES cell is derived from a frozen or cryopreserved inbred C57BL/6 embryo, and wherein the ES cell is able to proliferate in an undifferentiated state for more than 1 year when cultured in the presence of LIF on feeder cells, and wherein the ES cell is able to generate live pups at a frequency of at least 5%, at least 10%, at least 15%, or at least 20% in a tetraploid embryo complementation assay. In some embodiments, the embryo comprises a genetic modification, for example, a genetic modification as described herein.

### DETAILED DESCRIPTION OF CERTAIN EMBODIMENTS

Described herein are inbred C57BL/6 ES cells of high developmental capacity derived from frozen or cryopreserved C57BL/6 embryos, such as inbred C57BL/6 ES cells of high developmental capacity derived from frozen C57BL/6J embryos or cryopreserved C57BL/6J embryos. The development of these lines is surprising, in that it has been presumed in the field that freezing or cryopreservation of cells and embryos has a detrimental effect on their viability and developmental capacity and that ES cell lines derived from frozen or cryopreserved embryos exhibit inferior developmental capacity, compared with the developmental capacity of ES cell lines derived from fresh embryos. The inbred C57BL/6 ES cells, cell lines, and cell populations provided herein exhibit a developmental capacity sufficient to generate live pups after tetraploid blastocyst complementation.

It is often desirable to generate a transgenic mouse on a C57BL/6 background, because the C57BL/6 genome has been extensively characterized. Backcrossing a transgenic line from a non-C57BL/6 chimera (e.g., from a chimera derived from the commonly used inbred 129 ES cells or an outbred ES cell line) to produce a C57BL/6 line having the desired genetic modification has major disadvantages. For example, the resulting mice are a random genetic mixture of two different mouse strains (e.g., 129×C57BL/6), and if a pure C57BL/6 background is required, extensive inbreeding of the backcrossed mice is required, which can take years to complete (e.g., 10 generations of matings with C57BL/6 mice). Further, even after extensive inbreeding, the resulting backcrossed lines often carry uncharacterized genetic traits of the non-C57BL/6 line, making the phenotypic characterization of a genetic perturbation difficult. Direct generation of transgenic or knockout mice using C57BL/6 ES cells would eliminate the disadvantages, delays and costs associated with backcrossing strategies.

Recently, large-scale knockout mouse projects have been established by the National Institutes of Health (NIH) to generate new knockout mice in the C57BL/6 genetic background (Austin et al. 2004). However, only few ES cell lines are currently available from C57BL/6 and C57BL/6N mouse strains (Kontgen et al. 1993, Pettitt et al. 2009) and available inbred C57BL/6 ES cell lines do not exhibit the same developmental capacity as their outbred or 129 inbred counterparts. For example, no inbred C57BL/6 ES cell line is currently available that reliably generates live pups via tetraploid embryo complementation, and chimeras generated via diploid blastocyst injection with inbred C57BL/6 ES cells are often low-contribution chimeras whose germline does not comprise a cell derived from the injected C57BL/6ES cells.

The most commonly used approach to generate chimeric mice from ES cells is blastocysts microinjection, where one or more ES cells, for example, ES cells carrying a targeted genetic manipulation, are microinjected into a host blastocyst, which is then transferred to a foster mouse to generate ES cell-chimeric pups. In conventional blastocyst injections, a normal, diploid host blastocyst is injected with the desired ES cell population and both the injected ES cells and the inner cell mass (ICM) cells of the host blastocyst contribute to tissues of the chimeric mouse developing from such a blastocyst. Injected blastocysts can be transferred to foster mice to generate chimeric pups. If blastocyst injection is performed to produce a mouse line from the injected ES cells, the chimeric mice obtained from the blastocyst can be bred and, if the ES cell contributed to the germline of the chimeric mouse, ES-cell derived offspring can then be used to found an ES cell-derived mouse line, such as a line carrying a desired genetic modification. Host blastocysts used for the production of chimeric mice are typically derived from a mouse strain that is different from the strain of the injected ES cell line, which facilitates recognition of chimeric mice and offspring of chimeric mice by phenotype (e.g. coat color) or genotype of chimeric offspring. Same-strain injections are feasible as well, but require more effort in characterizing the generated mice.

For example, mice carrying a desired genetic modification can be propagated by breeding this “germline” chimera, thus generating pups in which all cells carry one ES cell derived set of chromosomes. Genetic screening, for example, for a genetic modification introduced into the ES cells, and further breeding of the desired offspring of a germline chimera can then be performed to create a transgenic mouse strain.

Tetraploid blastocyst complementation is a special type of blastocyst injection approach, where diploid ES cells are injected into a tetraploid host blastocyst. Such tetraploid host blastocysts are typically generated by fusing the blastomeres of a diploid 2-cell stage embryo, thus generating an embryo made of one tetraploid cell. Tetraploid embryos are then cultured to the blastocyst stage and injected with diploid ES cells. The tetraploid cells of the host blastocyst form the extraembryonic tissues, such as the placenta, but show no significant contribution to tissues of the developing mouse. Accordingly, virtually all cells, including germ cells, of mice generated by tetraploid blastocyst complementation are derived from the injected diploid ES cells. Such mice can directly be used for phenotypic characterization, or as founder animals for a transgenic mouse strain.

### Mouse Inbred C57BL/6 ES Cells, Cell Populations, and Cell Lines.

As described herein, inbred C57BL/6 ES cell lines of high developmental capacity have been produced, including inbred C57BL/6 ES cell lines of high developmental capacity that are derived from frozen or cryopreserved inbred C57BL/6 embryos, such as, from frozen or cryopreserved C57BL/6J embryos. The term “high developmental capacity,” as used herein, refers to a developmental capacity of an ES cell that is sufficient to support the generation of live pups at term after tetraploid blastocyst complementation. In some embodiments, the term refers to a developmental capacity sufficient to generate at least 5%, at least 10%, at least 15%, at least 20%, or at least 25% live pups at term after tetraploid blastocyst complementation, as measured by the ratio of live pups at term to the number of complemented tetraploid blastocysts transferred to pseudopregnant female. In some embodiments, the term refers to a developmental capacity sufficient to support the generation of adult mice via tetraploid embryo complementation, for example, of at least 5%, at least 10%, at least 15%, at least 20%, or at least 25% of adult mice from tetraploid blastocyst complementation as measured by the ratio of mice surviving to adulthood over the number of complemented tetraploid blastocysts transferred into a pseudopregnant female. In some embodiments, the term refers to a developmental capacity sufficient to support development of live pups after tetraploid embryo complementation, and sufficient to support germline contribution after diploid blastocyst complementation (e.g., contribution of more than 50%, more than 60%, more than 70%, more than 80%, more than 90%, more than 95%, more than 98%, more than 99%, or of 100% of the germline of the chimera, as measured by ES-cell derived pups over host blastocyst-derived pups obtained from a chimera).

The inbred mouse strain represents one of the most important milestones in the history of mouse genetics, and it revolutionized studies in cancer research, tissue transplantation, immunology, neuronal science and physiology. An inbred mouse strain is defined as a strain that has been maintained for more than 20 generations of intra-strain matings. In some embodiments, an inbred mouse strain is a strain that is produced using at least 20 consecutive generations of sister×brother or parent×offspring matings, or that is traceable to a single ancestral pair in the 20th or a subsequent generation. Typically, an inbred mouse strain is a strain that is homozygous at essentially all genetic loci except for the sex determining loci (Altman and Katz 1979).

The C57BL/6 inbred mouse strain is one of the most commonly inbred mouse strains used in biomedical research and has far surpassed 20 generations of inbreeding. As a result, C57BL/6 mice are genetically homozygous at virtually all of their genomic loci, except for their sex-determining loci. As with other inbred mouse strains, the C57BL/6 strain has a unique set of characteristics that sets it apart from all other inbred strains. Several substrains of the inbred C57BL/6 mouse strain are known to those of skill in the art, including, for example, the C57BL/6J and C57BL/6N substrains.

An inbred embryo, (e.g., an inbred C57BL/6 embryo) is an embryo that is generated as a result of inbred mating, for example, of two mice of the same inbred strain, for example, an embryo generated as a result of a C57BL/6×C57BL/6 mating. In some embodiments, an inbred embryo is an embryo, for example, an inbred C57BL/6J embryo, that is generated as a result of an inbred mating, for example, from two mice of the same inbred substrain, for example, an embryo generated as a result of a C57BL/6J×C57BL/6J mating. In some embodiments, an inbred embryo is an embryo, for example, an inbred C57BL/6 embryo, is an embryo that is generated as a result of an inbred mating, for example, from two mice of different substrains of an inbred strain, for example, an embryo generated as a result of a C57BL/6J×C57BL/6N mating.

Two novel inbred C57BL/6 ES cell lines derived from cryopreserved C57BL/6 embryos, designated MK6 and MK6V, respectively, are provided herein. These ES cell lines have been derived from mouse inbred C57BL/6J cryopreserved embryos. Both of these lines exhibit a normal male (XY) genotype and undifferentiated normal morphology during long term culture on feeder cells. Some embodiments provide a cell, a plurality of cells, and/or a cell population of the MK6 ES cell line. Some embodiments provide a cell, a plurality of cells, and/or a cell population of the MK6V ES cell line. Some embodiments provide a cell or a cell population derived from the MK6 ES cell line or the MK6V ES cell line. Some embodiments provide a pluripotent cell, plurality of cells, and/or cell population derived from the MK6 ES cell line or the MK6V ES cell line. Some embodiments provide a differentiated cell, plurality of cells, and/or cell population derived from the MK6 ES cell line or the MK6V ES cell line.

The inbred C57BL/6 mouse ES cells of high developmental capacity described herein, for example, inbred C57BL/6J mouse ES cells of the MK6 or the MK6V ES cell lines, are pluripotent cells. The term “pluripotent,” as used in the context of ES cells, describes the developmental capacity of a cell or cell population and refers to cells that have the capacity to differentiate into cells of any of the three germ layers (endoderm, mesoderm, and ectoderm). A pluripotent cell has the potential to give rise to any fetal or adult cell type. However, pluripotent cells cannot contribute to extraembryonic tissue, such as, the placenta. This distinguishes pluripotent cells from totipotent cells, which can give rise to all fetal, adult, and extraembryonic cell types.

Some embodiments are ES cell lines derived from frozen or cryopreserved inbred C57BL/6 embryos, for example, from frozen or cryopreserved inbred C57BL/6 embryos, for example, C57BL/6J embryos. The term “frozen,” as used herein in the context of embryos, refers to an embryo that has been cooled to a temperature below the freezing point of water, but not below a temperature of about −35° C. In some embodiments, a frozen embryo is an embryo that has been cooled to a temperature of −7° C. In some embodiments, a frozen embryo is an embryo that has been cooled to a temperature of −20° C. In some embodiments, a frozen embryo is an embryo has been cooled to a temperature of −35° C. The term “cryopreserved,” refers to an embryo that has been cooled to a temperature below −35° C. In some embodiments, a cryopreserved embryo is an embryo that has been cooled to a temperature of about −40° C. or less, of about −60° C. or less, or of about −80° C. or less. In some embodiments, a cryopreserved embryo is an embryo that has been cooled to the temperature of liquid nitrogen (LN2), for example, by incubation of the vessel or straw harboring the embryo in a liquid nitrogen tank.

Embryo freezing and embryo cryopreservation are routine methods used in modern mouse colony management and are well known to those of skill in the art. For more than 20 years, scientists have successfully stored and retrieved viable mouse embryos by freezing and cryopreservation. In theory, living embryos may be stored for an infinite period of time and then used to regenerate a colony of live animals Embryos can be stored at different temperatures, but for long-term storage, liquid nitrogen temperature (about −196° C.) is preferable. Frozen or cryopreserved embryos are metabolically inactive, for example, in that enzyme function and DNA mutations do not occur. Embryos can be thawed after freezing or cryopreservation and subsequently continue their development and give rise to a live mouse. However, freezing and cryopreservation have been reported to be associated with decreased viability and developmental capacity of the embryos. For an overview of freezing and cryopreservation methods and materials, see, e.g., Brigid Hogan, Manipulating the mouse embryo, Cold Spring Harbor Laboratory Press; 2nd edition (Nov. 1, 1994), ISBN 0879693843; Andreas Nagy et al., Manipulating the mouse embryo: A Laboratory Manual, Cold Spring Harbor Laboratory Press; 3rd edition (Dec. 15, 2002), ISBN 0879695919 and Paul Wassarman and Philippe Soriano, Guide to Techniques in Mouse Development, Part A: Mice, Embryos, and Cells (Methods in Enzymology), Academic Press; 1 edition (Sep. 9, 2010), ISBN 0123848830; all of which are incorporated herein by reference in their entirety, in particular, the passages that disclose freezing and cryopreservation methods and materials useful for such methods.

In some widely used freezing and/or cryopreservation methods, embryos are collected, equilibrated in a solution containing a cryoprotectant, placed into a vessel or special freezing straw that can withstand the desired temperature, and then cooled to the desired temperature. In some embodiments, the cooling comprises a step of controlled cooling at a specific cooling rate, for example, at a rate within the range of about 0.1° C.-about 10° C. per minute. In some embodiments, the controlled cooling is at a rate of about 0.1° C. per minute, about 0.2° C. per minute, about 0.3° C. per minute, about 0.4° C. per minute, about 0.5° C. per minute, about 0.6° C. per minute, about 0.7° C. per minute, about 0.8° C. per minute, about 0.9° C. per minute, about 1° C. per minute, about 1.25° C. per minute, about 1.5° C. per minute, about 1.75° C. per minute, about 2° C. per minute, about 2.5° C. per minute, about 3° C. per minute, about 4° C. per minute, about 5° C. per minute, about 6° C. per minute, about 7° C. per minute, about 8° C. per minute, about 9° C. per minute, or about 10° C. per minute.

In some embodiments, the cooling involves a controlled cooling step within a temperature range of about 4° C.-about −80° C. In some embodiments, the controlled cooling step involves cooling from about 0° C.-about −40° C., or from about −7° C.-about −35° C. For example, some embodiments, the cooling involves a controlled cooling step from about −7° C. about −35° C. at a rate of about 0.5° C. per minute. In some embodiments, the cooling involves an initial, controlled cooling step, for example, from about −7° C.-about −35° C., and a second, uncontrolled cooling step, for example, from about −35° C. to about −80° C. or to the temperature of liquid nitrogen. In some embodiments, the uncontrolled cooling step involves incubating the vessel or straw containing the embryos at the desired ends temperature, for example by placing the embryos into a −80° C. freezer, or into a liquid nitrogen tank. In some embodiments, an embryo is cooled in a controlled cooling step to a target temperature of about −35° C. to −80° C. and then immediately placed into storage in liquid nitrogen (about −196° C.).

In some embodiments, an embryo is equilibrated in a solution containing a cryoprotectant before it is frozen or cryopreserved. Chemical cryoprotectant compounds are well known to those of skill in the art and some cryoprotectants are described, for example, in the Hogan; Nagy et al. and Wassarman and Soriano references cited herein, the contents of which are incorporated herein by reference. Typically, relatively low molecular weight, cell membrane-permeable cryoprotectant compounds are used for freezing mouse embryos. The term cell-membrane-permeable refers to the ability of the cryoprotective agent to cross the cell membrane and enter a living cell. Non-limiting examples of useful cryoprotectants are glycerol, dimethylsulfoxide (DMSO), ethylene glycol, and propylene glycol (1-2, propanediol). Cryoprotectant solutions are frequently supplemented with a sugar, for example, with glucose, sucrose, or mannose, to enhance freezing and thawing osmotic equilibration phenomena. Useful cryoprotectants are highly water-soluble and are typically employed in a concentration range of about 0.5M-about 2.0M. In some embodiments, the embryo is subjected to a brief period of equilibration in the cryoprotectant solution, for example, for a time of about 0.5 min-about 10 min, preferably for a time between 1-2 min, prior to initiation of the cooling procedure.

Without wishing to be bound by theory, it is believed that cryoprotective agents like DMSO and glycerol improve the survival rate of frozen or cryopreserved cells by decreasing the temperature at which ice forms. For example, it is believed that some widely used cryoprotectants lower the freezing point of embryonic cells (for example, to −4 to −6° C.), and permit controlled dehydration of the cells. It is further believed that the movement of water into and out of the cell during cooling determines the dynamics of intracellular ice formation and cell survival. It is believed that prolonged exposure to high concentrations of cryoprotectants is toxic for mouse embryos. Accordingly, in some embodiments, a freezing protocol is used that avoids the formation of intracellular ice within the cells of an embryo to be frozen, while minimizing cell damage resulting from prolonged exposure to cryoprotectant agents or other agents in the cryoprotectant solution. See “The Cryopreservation of Mouse Embryos” by Taconic Farms, Inc., www.taconic.com/wmspage.cfm?parm1=321.

In some embodiments, an embryo to be frozen or cryopreserved is first equilibrated in cryoprotectant solution, then cooled to about −5° to about −7° C., and subsequently the extracellular medium is induced to freeze by a process referred to as seeding prior to the initiation of a controlled cooling step. In some embodiments, the seeding temperature is just below the freezing temperature of the medium or solution in which the embryo is frozen or cryopreserved. In some embodiments, seeding is achieved by agitating the freezing vessel or straw at the seeding temperature, for example, by gripping the vessel or straw with cooled forceps. The critical process of seeding prevents the potentially damaging effects of uncontrolled crystallization of the freezing media. The optimal seeding temperature is dependent on the specific cryoprotectant solution employed.

Embryos can be frozen or cryopreserved at any preimplantation stage, for example, at the 1-cell stage (e.g., after fertilization), the 2-cell stage, the 4-cell stage, the 8-cell stage, the 16-cell stage, the morula stage, or the blastocyst stage. Any such stage is useful for the ES cell derivation methods described herein.

Frozen or cryopreserved embryos can be stored for extended periods of time, (e.g., for at least one month, at least two months, at least three months, at least six months, at least one year, at least two years, at least three years, at least five years, or at least 10 years) without losing their developmental capacity. For example, in some embodiments, pluripotent ES cells with high developmental capacity are derived from inbred C57BL/6 embryos that have been frozen or cryopreserved for an extended period of time as described above.

Derivation of ES cells from a frozen or cryopreserved embryo involves thawing the embryo. Various thawing procedures, including controlled rate and endpoint procedures, are known to those of skill in the art. The thawing procedure employed for thawing a frozen or cryopreserved embryo depends, among other factors, on the specific freezing protocol and choice of cryoprotectant. In some embodiments, frozen or cryopreserved mouse embryos are rapidly warmed from their storage temperature, for example, from about −35° C., about −40° C., about −60° C., about −70° C., about −80° C., or about −196° C., to the target temperature. In some embodiments, the target temperature is room temperature (e.g., about 20-25° C.), or the desired culture temperature (e.g. about 35-39° C.). In some embodiments, rapid thawing of a frozen or cryopreserved embryo is achieved by placing the freezing vessel or straw containing the embryo into an appropriately heated water bath. In some environments, frozen or cryopreserved mouse embryos are warmed at a controlled rate to a target temperature, for example at a rate of about 10° C. to about 20° C. per minute.

In some embodiments, a frozen or cryopreserved embryo is incubated in an appropriate culture medium, for example PBS or M2 medium, after thawing in order to remove the cryoprotectant. In some embodiments, stepwise dilution and cryoprotectant removal is performed, rather than direct dilution. For example, in some embodiments, if the cryoprotectant solution contains 1.5M glycerol, an embryo may be transferred from the cryoprotectant solution to culture media through a series of glycerol dilutions (e.g. 1.25M, 1M, 0.75M, 0.5M, 0.25M, 0M glycerol in culture media).

Some embodiments provide a cell, a plurality of cells and/or a cell population of an inbred C57BL/6 ES cell line derived from a frozen or cryopreserved inbred C57BL/6 embryo. Some embodiments provide a cell, a plurality of cells and/or a cell population derived from an inbred C57BL/6 ES cell line derived from an inbred frozen or cryopreserved inbred C57BL/6 embryo. In some embodiments, cells of the ES cell line derived from frozen or cryopreserved C57BL/6 embryo exhibit a high developmental capacity.

The term “developmental capacity,” as used herein, refers to the ability of a cell or a plurality of cells to give rise to different fetal or adult cell types. ES cells, including the inbred C57BL/6 ES cells provided herein, are pluripotent cells that have the capability to give rise to all fetal or adult cell types. Some ES cells, however, even though they are pluripotent, exhibit biased differentiation capabilities, such as, a preference to differentiate into neuronal lineages, and a lower rate or inefficient state transition into endoderm lineages. Such differentiation bias can manifest, for example, in skewed ES-cell contribution to certain tissues of diploid chimeric mice. For example, some chimeric mice derived from ES cells with biased differentiation capabilities exhibit high contribution to brain tissue, but only low or no contribution to the germ line. On the other hand, some ES cells show balanced differentiation capabilities but only contribute at low frequency to chimeric mice. It is believed that the injected ES cells compete with the ICM cells of the host blastocysts during development and that ES cells of low developmental capacity can be outcompeted by diploid host blastocyst ICM cells, either in general or in specific tissues. On the other hand, ES cells of high developmental capacity can outcompete the ICM cells of a diploid host blastocyst, resulting in chimeric mice in which the majority of cells are derived from the injected ES cells and not from the host blastocyst. If an ES cell or cell line is to be used for the generation of genetically modified mice, high developmental capacity is preferred.

It has been reported that inbred ES cells and ES cell lines generally exhibit a lower developmental capacity than their outbred counterparts. See, for example Eggan et al., PNAS, 2001 (May) 6209-6214; incorporated herein by reference in its entirety, particularly those passages disclosing the developmental capacity of various inbred and outbred ES cell lines, methods of assessing ES cell developmental capacity, and methods to generate chimeric and ES cell derived mice by diploid and tetraploid blastocyst complementation. The most stringent test for the developmental capacity of an ES cell is the tetraploid blastocyst complementation assay, which is well known to those of skill in the art (see, e.g., Eggan et al., 2001). In this assay, an ES cell or a plurality of ES cells is injected into a tetraploid host blastocysts. The tetraploid host blastocyst can contribute to extra embryonic tissues, such as, the placenta, but does not give rise to fetal or adult cell types. Accordingly, a tetraploid blastocyst alone cannot give rise to a live pup at birth, because it cannot generate the required fetal tissues. When injected with a diploid ES cell or a plurality of diploid ES cells, however, this “complemented” tetraploid blastocyst can give rise to a live pup, if the injected ES cells exhibit a high developmental capacity. Tetraploid blastocyst complementation is a more stringent test for developmental capacity than the derivation of chimeric mice after diploid blastocyst injection, because any defect in ES cell developmental capacity cannot be compensated by ICM cells of the host blastocyst. Further, pups born after tetraploid blastocyst complementation frequently die shortly after birth; this postnatal mortality is particularly pronounced in pups derived from tetraploid blastocysts complemented with inbred ES cells. If an ES cell, or a plurality of ES cells gives rise to a live pup after tetraploid blastocyst complementation, the ES cell or plurality of ES cells exhibit a high and unbiased developmental capacity. Survival of such tetraploid complementation pups to adulthood is a further indicator of high developmental capacity.

Some embodiments provide inbred ES cells, ES cell populations, and ES cell lines of high developmental capacity derived from frozen inbred embryos or cryopreserved inbred embryos, such as from frozen C57BL/6 embryos or cryopreserved C57BL/6 embryos, and having a developmental capacity to generate at least 5% to at least 20% live pups after tetraploid blastocyst complementation. In some embodiments, the high developmental capacity of the ES cells, ES cell populations, or ES cell lines provided herein manifests in the ability to generate at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, or at least 20% of live pups after tetraploid blastocyst complementation.

In some embodiments, the ES cells, ES cell population, or ES cell line provided has been derived from an inbred mouse C57BL/6J frozen or cryopreserved embryo. In some embodiments, the ES cell line exhibits a normal male (XY) genotype and undifferentiated normal morphology during long term culture on feeder cells. In some embodiments, the inbred C57BL/6 ES cell, ES cell population, or ES cell line of high developmental capacity provided herein has substantially the same developmental capacity as ES cell line MK6, or as ES cell line MK6V. In some embodiments, the ES cells, ES cell populations, or ES cell lines have the ability to generate live pups after tetraploid embryo complementation that survive to adulthood. In some embodiments, the cells, cell populations, or cell lines provided herein have the ability to generate fertile live pups.

Some embodiments provide a non-human pre-implantation embryo comprising an inbred C57BL/6 mouse ES cell of high developmental capacity or a plurality of such mouse ES cells as provided herein. For example, some embodiments provide a diploid host blastocyst that comprises an inbred mouse C57BL/6 mouse ES cell of high developmental capacity as provided herein in the blastocoele. In other embodiments, a tetraploid blastocyst is provided that comprises an inbred C57BL/6 mouse ES cell of high developmental capacity as provided herein in the blastocoele. In general, preimplantation embryos as provided herein are generated by injecting an ES cell as provided herein into the blastocoele of a diploid or a tetraploid host blastocyst. Methods for blastocyst injection are well-known to those of skill in the art (see, e.g., Brigid Hogan, Manipulating the mouse embryo, Cold Spring Harbor Laboratory Press; 2nd edition (Nov. 1, 1994), ISBN 0879693843; Andreas Nagy et al., Manipulating the mouse embryo: A Laboratory Manual, Cold Spring Harbor Laboratory Press; 3rd edition (Dec. 15, 2002), ISBN 0879695919; and Paul Wassarman and Philippe Soriano, Guide to Techniques in Mouse Development, Part A: Mice, Embryos, and Cells (Methods in Enzymology), Academic Press; 1 edition (Sep. 9, 2010), ISBN 0123848830; all of which are incorporated herein in their entirety by reference, in particular those passages disclosing embryo manipulation techniques. In some embodiments, the host blastocyst is of a different genetic background then the injected C57BL/6 ES cell(s) of high developmental capacity.

Some embodiments provide a mouse ES cell that is derived from an inbred C57BL/6 embryo, for example, from an inbred C57BL/6J embryo, and that exhibits a high developmental capacity. In some embodiments, the mouse ES cell, or a pluripotent derivative thereof is able to generate more than 5% live pups, more than 10% live pups, more than 15% live pups, or more than 20% live pups (expressed as the average of live pups at term/tetraploid complemented embryos transferred into a pseudopregnant host) after tetraploid blastocyst complementation. In some embodiments, the complemented tetraploid blastocysts are cBrd/cBrd/cr or BALB/c blastocysts. In some embodiments, the inbred C57BL/6 ES cell provided has been derived from a frozen embryo. In some embodiments, the inbred C57BL/6 ES cell provided has been derived from a cryopreserved embryo. In some environments, the inbred C57BL/6 ES cell provided has been derived from an embryo that has been frozen or cryopreserved at the 1-cell stage, at the 2-cell stage, at the 8 cell stage, at the 16 cell-stage, at the morula stage, or at the blastocyst stage. In some embodiments, the inbred C57BL/6 ES cell provided has been derived from a frozen or cryopreserved embryo that has been frozen or cryopreserved for at least 3 months, at least 6 months, at least 1 year, at least 2 years, or at least 3 years.

In some embodiments, the inbred C57BL/6 ES cell provided exhibits a normal karyotype. The term “karyotype” refers to the number and appearance of chromosomes in a given cell. The number of chromosomes in a normal, diploid mouse cell is 40, including the two sex chromosomes (40,XX or 40,XY). The term “normal karyotype,” as used herein, refers to the number and appearance of chromosomes found in a normal, diploid mouse cell. A cell exhibiting either a numerical or a structural chromosomal abnormality does not exhibit a normal karyotype. For example, an aneuploid mouse cell, comprising an aberrant number of chromosomes (any number other than 40, e.g. due to a trisomy), or an euploid cell (comprising 40 chromosomes) but with at least one of them of aberrant appearance, for example, due to a structural abnormality (e.g., deletion, duplication, translocation, break, fusion), are not exhibiting a normal karyotype. Methods for determining cellular karyotypes are well known to those of skill in the art and some exemplary karyotyping methods involving the preparation and analysis of metaphase spreads are described herein.

In some embodiments, the inbred C57BL/6 ES cell provided exhibits a normal 40, XY karyotype and produces male pups after tetraploid embryo complementation. In some embodiments, the inbred C57BL/6 ES cell provided exhibits a normal 40, XX karyotype and produces female pups after tetraploid embryo complementation. In some embodiments, the inbred C57BL/6 ES cell provided exhibits a 39, X0 karyotype, for example, after loss of the Y chromosomes, and produces female pups after tetraploid embryo complementation. Loss of the Y chromosome is not unusual in ES cells and often ES cell populations that are predominantly karyotypically normal 40,XY comprise a small percentage of cells that have lost the Y chromosome. In some embodiments, inbred C57BL/6 ES cells of a 39,X0 karyotype are isolated, for example, by single-cell subcloning, and the subclones are cultured under the same conditions as the parent ES cell population. In some embodiments, 39,X0 ES cells isolated from an ES cell population provided herein are injected into tetraploid blastocysts, giving rise to a fertile, female pup.

The term subcloning refers to the isolation of a single cell from a population of cells, for example, of a single MK6 or MK6V ES cell from a population of MK6 or MK6V ES cells, respectively, and the propagation of that single cell to a cell population, or subclone, that can be traced back to the single, isolated cell. Subcloning is a method useful for isolating and propagating cells of an identical, desired phenotype of genotype from a mixed population of cells. For example, in some embodiments, subcloning is performed to isolate a karyotypically normal subclone, a karyotypically abnormal subclone (e.g., a 39,X0 subclone), or a subclone comprising a desired genetic modification from a mixed parent population of cells, for example, of MK6 or MK6V ES cells.

Any given ES cell population will comprise a small percentage of karyotypically abnormal cells. An ES cell population is deemed to exhibit a normal karyotype if the majority of cells within that population exhibits a normal karyotype. Accordingly, in some embodiments, an ES cell population is deemed to exhibit a normal karyotype if more than 50%, more than 60%, more than 70%, more than 75%, more than 80%, more than 85%, more than 90%, more than 95%, more than 98%, more than 99%, more than 99.5%, or more than 99.9% of cells exhibit a normal karyotype.

### ES Cell Derivatives

Some embodiments provide pluripotent and differentiated cells derived from the inbred C57BL/6 ES cell lines provided, for example, from the MK6 ES cell line or the MK6V ES cell line provided or from the inbred C57BL/6 ES cell lines provided that are derived from frozen or cryopreserved embryos and generate at least 5% or more live pups after tetraploid embryo complementation. In some embodiments, a cell derived from a stem cell line provided herein, also referred to herein as a “derivative” is a pluripotent cell obtained by culturing a cell of a stem cell line provided herein under conditions suitable for stem cell maintenance and self renewal. For example, an ES cell derived from or an ancestor of which has been derived from the MK6 cell line or the MK6V ES cell line by single-cell subcloning, for example, by single cell subcloning of a 40,XY subclone, or of a 39,X0 subclone, is a derivative of the respective ES cell line.

In some embodiments, an ES cell derivative as provided herein is a pluripotent cell derived from an ES cell or ES cell line as provided herein. For example, in some embodiments, an ES cell derivative is provided that itself is an ES cell, for example, a subcloned ES cell, or a genetically modified ES cell. Methods for the derivation of subclones and for generating genetically modified ES cells from established ES cell lines are well known to those of skill in the art and include, for example, the methods and protocols described in Kursad Turksen, Embryonic Stem Cell Protocols: Volume I: Isolation and Characterization (Methods in Molecular Biology), Humana Press; 2nd edition (Feb. 15, 2006), ISBN 1588294986; Kursad Turksen, Embryonic Stem Cell Protocols: Volume II: Differentiation Models (Methods in Molecular Biology), Humana Press; 2nd edition (Feb. 1, 2006), ISBN 1588297845; Alexandra L. Joyner, Gene Targeting: A Practical Approach, Oxford University Press, USA; 2 edition (Feb. 15, 2000), ISBN 019963792X; Ralf Kühn and Wolfgang Wurst, Gene Knockout Protocols, Humana Press; 2nd ed. edition (Mar. 27, 2009), ISBN 1934115266; Eric B. Kmiec, Gene Targeting Protocols (Methods in Molecular Biology), Humana Press; 1 edition (Jan. 15, 2000), ISBN 0896033600; all of which are incorporated in their entirety herein by reference, in particular, those passages that disclose methods for the derivation of native or genetically modified pluripotent and differentiated cells from ES cells and cell lines.

In some embodiments, a genetically modified pluripotent cell, for example, an ES cell is provided that is a derivative of an ES cell or cell line herein. In some embodiments, the genetically modified derivative cell or cell population is obtained by contacting a cell of the parent cell line, for example, a cell of the MK6 or the MK6V cell line, with a nucleic acid comprising a heterologous nucleic acid construct, for example, a gene targeting construct, a knockout construct, an expression construct, or a viral construct, and isolating and/or subcloning a cell that has the nucleic acid construct, or a part thereof, stably inserted into its genome. In some embodiments, the nucleic acid construct comprises a selection cassette, for example, an expression cassette conferring resistance to antibiotic, or expressing a selectable marker, for example, a fluorescent protein. Suitable antibiotics for the selection of genetically modified ES cells are well known in the art and include for example, neomycin, G418, hygromycin, blasticidin, and puromycin. In some embodiments, a genetically modified pluripotent cell, for example, genetically modified ES cell, is derived from an ES cell or an ES cell population provided herein by contacting a cell of the ES cell population with a nucleic acid construct comprising a selection cassette and subsequent selection, for example by contacting the cell population with the appropriate antibiotic, or by selecting cells based on the expression of a selectable marker, for example by fluorescence activated cell sorting (FACS) of a cell population comprising cells expressing a fluorescent protein from the nucleic acid construct. In some embodiments, the isolated genetically modified ES cell is then subcloned to generate a genetically modified ES cell population or ES cell line.

In some embodiments, a cell derived from an inbred C57BL/6 ES cell line of high developmental capacity as provided herein is a differentiated cell obtained by culturing an inbred C57BL/6 ES cell of high developmental capacity provided herein under conditions inducing ES cell differentiation. Methods and conditions for inducing ES cell differentiation are well known to those of skill in the art, and include, for example, methods and conditions described in Kursad Turksen, Embryonic Stem Cell Protocols: Volume I: Isolation and Characterization (Methods in Molecular Biology), Humana Press; 2nd edition (Feb. 15, 2006), ISBN 1588294986; Kursad Turksen, Embryonic Stem Cell Protocols: Volume II: Differentiation Models (Methods in Molecular Biology), Humana Press; 2nd edition (Feb. 1, 2006), ISBN 1588297845; Alexandra L. Joyner, Gene Targeting: A Practical Approach, Oxford University Press, USA; 2 edition (Feb. 15, 2000), ISBN 019963792X; Ralf Kühn and Wolfgang Wurst, Gene Knockout Protocols, Humana Press; 2nd ed. edition (Mar. 27, 2009), ISBN 1934115266; Eric B. Kmiec, Gene Targeting Protocols (Methods in Molecular Biology), Humana Press; 1 edition (Jan. 15, 2000), ISBN 0896033600; all of which are incorporated in their entirety herein by reference, in particular, those passages that disclose methods for the derivation of native or genetically modified differentiated cells from ES cells and cell lines.

For example, in some embodiments a differentiated cell derived from an ES cell as provided herein is a differentiated cell in a mouse generated by diploid or tetraploid blastocyst complementation with an ES cell as provided herein, wherein the cell can be traced back to an injected ES cell. In some embodiments, a differentiated cell derived from an ES cell as provided herein is a cell that has been obtained by in vitro differentiation of an ES cell as provided herein. Methods and protocols for in vitro differentiation of ES cells are well known to those of skill in the art and includes, for example the methods and protocols for in vitro differentiation described in the references immediately above. The methods employed to generate a specific cell type by differentiation of ES cells will depend of course on the desired differentiated cell type.

Derivation of Inbred C57BL/6 ES Cells from Frozen or Cryopreserved Embryos

Some embodiments provide methods to derive pluripotent inbred C57BL/6 ES cells of high developmental capacity from frozen or cryopreserved inbred C57BL/6 embryos. In some embodiments, methods for the derivation of inbred C57BL/6J ES cells of high developmental capacity from frozen or cryopreserved inbred C57BL/6J embryos are provided. In some embodiments, the method comprises a step of providing a frozen or cryopreserved, inbred C57BL/6 mouse embryo, for example, a frozen or cryopreserved, inbred C57BL/6J embryo. In some embodiments, the embryo has been frozen or cryopreserved for at least 3, at least, 6, at least 12, at least 24, or at least 36 months.

In some embodiments, the method comprises a step of thawing the embryo. In some embodiments, the frozen or cryopreserved embryo is thawed in M2 media. M2 media is an art-recognized embryo culture media well known to those of skill in the art. In some embodiments, the step of thawing the embryo involves subjecting the frozen or cryopreserved embryo to a controlled thawing procedure. In some embodiments, the step of thawing the embryo involves warming the frozen or cryopreserved embryo to a desired target temperature. In some embodiments, the target temperature is room temperature (20 to 25° C.). In some embodiments, the target temperature is a temperature appropriate for cell culture (e.g., 35-39° C., preferably about 37° C.).

In some embodiments, the method includes a step of culturing the embryo until the blastocyst stage. For example, in some embodiments, the embryo is an embryo that has been frozen at the eight cell stage, and after thawing the eight cell stage embryo is cultured until it reaches the blastocyst stage. In some embodiments, the embryo is cultured in KSOM-AA medium. KSOM-AA medium is an art recognized term describing an embryonic culture medium well known to those of skill in the art.

In some embodiments, the method includes a step of incubating the blastocyst developed from the frozen or cryopreserved embryo on feeder cells until an inner cell mass outgrowth is formed. In some embodiments, this incubating step involves culturing the blastocyst and any outgrowing cells with leukemia inhibitory factor (LIF) at a concentration sufficient for ES cell self renewal and maintenance of pluripotency. LIF is a cytokine of the interleukin six class that affects cell growth and development. In most mouse ES cell lines, withdrawal of LIF from ES cell culture media leads to differentiation of the cultured cells. While some mouse ES cells can be cultured in the absence of LIF, typically, mouse ES cells are cultured in media comprising sufficient amounts of LIF to maintain an undifferentiated state. LIF is commercially available or can be prepared by bacterial expression of the protein.

Methods for the preparation of LIF as well as recipes for ES cell culture media containing sufficient amounts of LIF for the maintenance of undifferentiated ES cell cultures are well known to those of skill in the art and include, for example, the methods and protocols described in Kursad Turksen, Embryonic Stem Cell Protocols: Volume I: Isolation and Characterization (Methods in Molecular Biology), Humana Press; 2nd edition (Feb. 15, 2006), ISBN 1588294986; Kursad Turksen, Embryonic Stem Cell Protocols: Volume II: Differentiation Models (Methods in Molecular Biology), Humana Press; 2nd edition (Feb. 1, 2006), ISBN 1588297845; Alexandra L. Joyner, Gene Targeting: A Practical Approach, Oxford University Press, USA; 2 edition (Feb. 15, 2000), ISBN 019963792X; Ralf Kühn and Wolfgang Wurst, Gene Knockout Protocols, Humana Press; 2nd ed. edition (Mar. 27, 2009), ISBN 1934115266; Eric B. Kmiec, Gene Targeting Protocols (Methods in Molecular Biology), Humana Press; 1 edition (Jan. 15, 2000), ISBN 0896033600; all of which are incorporated in their entirety herein by reference for disclosure of methods and recipes for ES cell culture and derivation.

In some embodiments, the step of incubating the blastocyst on feeder cells involves incubation of the blastocyst in ES cell culture medium. In some embodiments, the ES cell culture medium comprises Knockout DMEM (Invitrogen/GIBCO), knockout serum replacement (KSR, Invitrogen/GIBCO), 1,000 units/ml leukemia inhibitory factor (Chimicon), 0.1 mM 2-mercaptophenol (Sigma), 2 mM glutamax (Invitrogen), 1 mM sodium pyruvate, and/or 0.1 mM nonessential amino acids.

In some embodiments, the method comprises a step of disaggregating the inner cell mass outgrowth. In some embodiments, the disaggregating is achieved by enzymatic digestion of extracellular proteins, for example, of cell-cell adhesion proteins. In some embodiments the disaggregating is achieved by trypsinization. Methods and compositions useful for disaggregating ICM outgrowths for the derivation of ES cells are well known to those of skill in the art and the invention is not limited by this aspect. In some embodiments, the method comprises a step of subcloning or subculturing an ES cell population derived from the ICM outgrowth to establish an ES cell line.

The derivation of mouse ES cell lines from blastocysts is routine procedure, and the skilled artisan will be aware of methods, compositions, culture media, culture conditions, and materials useful for this procedure. Exemplary methods, compositions, culture media, culture conditions, and materials useful for the derivation of mouse ES cell lines from blastocysts are described in the references immediately above.

Derivation of Mice from Inbred C57BL/6 ES Cells and their Derivatives

Some embodiments provide methods of using inbred C57BL/6 ES cells for the generation of chimeric mice by diploid blastocyst injection, or fully ES cell derived mice by tetraploid blastocyst complementation. Some embodiments provide methods for to the use of inbred C57BL/6 ES cells, for example, C57BL/6J ES cells, for the generation of ES cell derived mice. In some embodiments, ES cells and methods are provided for the generation of ES cell derived mice via conventional blastocyst injection. In some embodiments, ES cells and methods are provided for the generation of ES cell derived mice by tetraploid blastocyst complementation. ES cells and methods provided herein are useful for the rapid generation of transgenic mice in the C57BL/6 background in a cost-effective and efficient manner.

Some embodiments provide a method for the generation of chimeric mice from an inbred C57BL/6 ES cell as provided herein by diploid blastocyst complementation as described in more detail elsewhere herein. For example, some embodiments provide methods for the generation of chimeric mice from an MK6 ES cell, an MK6V ES cell, or a transgenic derivative of either cell line.

Other embodiments provide a method for the generation of fully ES cell derived mice from an inbred C57BL/6 ES cell as provided herein by tetraploid blastocyst complementation as described in more detail elsewhere herein. For example, some embodiments provide methods for the generation of chimeric mice from an MK6 ES cell, an MK6V ES cell, or a transgenic derivative of either cell line.

The generation of mice by blastocyst complementation is a routine procedure well known to those of skill in the art. Methods and materials useful for carrying out blastocysts complementation procedures will be apparent to the skilled artisan and include, for example, methods and materials described in Brigid Hogan, Manipulating the mouse embryo, Cold Spring Harbor Laboratory Press; 2nd edition (Nov. 1, 1994), ISBN 0879693843; Andreas Nagy et al., Manipulating the mouse embryo: A Laboratory Manual, Cold Spring Harbor Laboratory Press; 3rd edition (Dec. 15, 2002), ISBN 0879695919; and Paul Wassarman and Philippe Soriano, Guide to Techniques in Mouse Development, Part A: Mice, Embryos, and Cells (Methods in Enzymology), Academic Press; 1 edition (Sep. 9, 2010), ISBN 0123848830; all of which are incorporated herein by reference in their entirety for disclosure of methods for generation of mice by diploid or tetraploid blastocyst complementation and materials useful for carrying out such methods.

In some embodiments, a blastocyst complementation procedure involves the step of providing a host blastocyst, for example a diploid or a tetraploid host blastocyst, and injecting an ES cell as provided herein into the blastocoele. In some embodiments, a single ES cell as provided herein is injected. In some embodiments, a plurality of ES cells as provided herein are injected, for example, 2-25 ES cells, or, preferably 5-10 ES cells per blastocyst. In some embodiments, the ES cell injection is carried out on a microscope stage using any micro manipulator to direct a holding pipette used for positioning the host blastocyst and an injection needle used for picking up the ES cell(s) from an ES cell population provided, and for injecting them into the blastocoele of the host blastocyst. In some embodiments, the injection needle has beveled tip allowing it to puncture the zona pellucida of the host blastocyst. In some embodiments, the tip of the injection needle is blunt. In some embodiments, a piezo drive is used to aid in puncturing the zona pellucida with the injection needle.

Methods and materials for the micromanipulation of mouse blastocysts, including, for example, the microinjection of ES cells into diploid or tetraploid host blastocysts, are well known to those of skill in the art and include, for example the methods described in the references immediately above, which are incorporated herein by reference for disclosure of these methods.

For the generation of life pups, for example, chimeric pups or fully ES cell derived pups, complemented blastocysts are transferred to the uterus of a pseudopregnant foster female. Methods and materials for the generation of pseudopregnant foster females and for embryo transfer into such fosters are well known to those of skill in the art and include, for example, the methods described in the references immediately above, which are incorporated herein by reference for disclosure of these methods.

Mice Generated from Inbred C57BL/6 ES Cells and their Derivatives

Some embodiments provide mice derived from the ES cell lines provided herein. In some embodiments, a mouse is provided that is derived from an inbred C57BL/6 ES cell provided herein, for example, from an inbred C57BL/6J ES cell line derived from a frozen or cryopreserved embryo, as provided herein (e.g. MK6 or MK6V), by conventional, diploid blastocyst injection. In some embodiments, the mouse is a chimeric mouse, in which some of the cells are derived from the host embryo while other cells of the mouse are derived from an injected inbred C57BL/6 ES cell. In some embodiments, the germline of the chimeric mouse comprises at least one cell that is derived from an injected inbred C57BL/6 ES cell. In some embodiments, the chimeric mouse produces offspring that is derived from an injected inbred C57BL/6 ES cell.

In some embodiments, a mouse is provided that is derived from an inbred C57BL/6 ES cell provided herein, for example, from an inbred C57BL/6J ES cell line derived from a frozen or cryopreserved embryo, as provided herein (e.g. MK6 or MK6V), by tetraploid blastocyst complementation. In some embodiments, essentially all cells of the mouse are derived from an inbred C57BL/6 ES cell injected into the tetraploid host blastocyst during tetraploid blastocyst complementation. In some embodiments the germline of mouse consists essentially of cells derived from an inbred C57BL/6 ES cell injected into the tetraploid host blastocyst.

The function and advantage of these and other embodiments of the present invention will be more fully understood from the example section below. The following example section is intended to illustrate the benefits of the present invention and to describe particular embodiments, but does not exemplify the full scope of the invention. Accordingly, it will be understood that the example section is not meant to limit the scope of the invention.

### EXAMPLES

### Example 1

### Animals and Embryos

### Animals

C57BL/6J mice were used to generate embryos for ES cell derivation. B6D2F1 (C57BL/6×DBA2 hybrid, F1 generation) mice were used as donors of host embryos, for example, for tetraploid blastocyst complementation experiments. The CD-1 (outbred) strain was used for embryos transfer recipient females. C57BL/6J, B6D2F1 (Jackson Laboratory, Bar Harbor, Me.) and CD-1 (Charles River Laboratories, Wilmington Mass.) mice were housed in AAALAC-accredited animal facility under specific pathogen-free (SPF) conditions.

### Embryos

C57BL/6J and B6D2F1 females mice were superovulated at 3 to 4 weeks old by intraperitoneal injection of 5 IU pregnant mare serum gonadotropin (PMSG, Sigma-Aldrich, St Louis, Mo.) and 46-48 hr later, injected 5 IU of human chorionic gonadotropin (hCG). Mice were then bred with C57BL/6J and B6D2F1 stud males, respectively. Peseudopregnant CD-1 mice were obtained by mating with vasectomized CD-1 stud males. Zygotes of C57BL/6 and B6D2F1 animals were collected at 0.5 d post coitum and cultured to the 2-cell stage in optimized medium (KSOM+AA; Chemicon International, Temecula, Calif.). Embryo cultures were overlaid with mineral oil (SAGE) in an incubator at 37° C., 5% CO₂.

### Example 2

### Cryopreservation of 8-Cell Stage Embryos from Inbred C57BL/6J Mice

The experiments described in this example were conducted to test whether pluripotent ES cell lines could be successfully derived from frozen or cryopreserved embryos after being subjected to a long-term frozen period of 3 years. The procedure described was derived for the cryopreservation of eight-cell stage mouse embryos as described in S. P. Leibo, 1986, “Genetic Engineering of Animals,” incorporated herein by reference in its entirety for disclosure of methods for cryopreservation and freezing of preimplantation embryos). C57BL/6J eight-cell stage embryos were collected and selected for cryopreservation by assessment of normal developmental progression.

The procedure employed was derived for the routine mouse embryos cryopreservation of eight-cell stage embryos: Ovulation was induced by intraperitoneal injection of gonadotropins into female mice, first with pregnant mare serum gonadotropin (PMS: Sigma-Aldrich, St Louis Mo.), followed 46-48 hours later by an injection of human chorionic gonadotropin (hCG: Sigma-Aldrich, St Louis Mo.). Females were mated with C57BL/6J males immediately after hCG injection. The following morning, zygotes (one-cell stage) were collected from oviducts and cultured in KSOM+AA medium (Chemicon International, Temecula, Calif.) for two-more days until they reached the eight-cell stage. Embryos exhibiting the normal morphology of eight-cell stage embryos were picked up with a micropipet and used for freezing.

Plastic ¼ ml and ½ ml insemination straws (Cat# A201, and #101, IMV international Corporation (Minneapolis, Minn.)) were used for freezing and before loading the embryos, ¼ ml straws were marked with graduation marks with a permanent marker at 1 cm and 7 cm from the open end. The C57BL/6J embryos were loaded into freezing straws according to the following steps:
(1) 1.0M Sucrose in M2 medium was aspirated into a freezing straw (Cat# A201, and #101 from IMV international Corporation, Minneapolis, Minn.) until the fluid column reached the 7 cm mark. Air was aspirated into the straw up to the 1 cm mark.
(2) 1.5 M CPA (Glycerol: Sigma) was aspirated into the straw up to the 1 cm mark, and then air was aspirated up to about ½ cm from the end of the straw.
(3) The recovered embryos from the KSOM+AA culture dish, which have been pre-selected eight-cell stage embryos, were rinsed twice in M2 medium. Embryos were then transferred into 1.5M glycerol/M2 media contained in a 35 mm Petri dish and allowed to settle in the solution.
(4) After equilibration, a set of 10-20 embryos were picked up from the glycerol solution and aspirated into the freezing straw. A small volume of sucrose or CPA was then aspirated into the straw.
(5) After aspiration of the embryos and other solutions, the straw was heat-sealed according to the manufacturer's recommendations on both ends.
(6) For freezing and cryopreservation, once all embryos were loaded into straws and then were placed into a controlled-rate freezer (Bio-Cool FTS). Freezing was carried out at a starting temperature of −7° C., and seeding was induced by touching each straw individually briefly with forceps cooled in liquid nitrogen (LN2). Seeding was verified by visual observation. The straws were cooled in the controlled-rate freezer from −7° C. to −35° C. at a rate of 0.5° C. per minute. For cryopreservation, the straws were dropped into liquid nitrogen after completion of the controlled freeze to −35° C.

### Example 3

### Derivation of ES Cells from Cryopreserved C57BL/6J Embryos

The eight-cell stage cryopreserved embryos were thawed in M2 medium and cultured in KSOM-AA medium (Specialty Media) overnight. Each straw was thawed individually by placing it in the horizontal position at room temperature air on a rack. The straw was thawed for 1 minute in air. The handle was removed and the straw was wiped. Then the straw was incubated in a vertical position, plug-end up, in a 37° C. water bath for 1 minute. The straw was removed from the water bath and wiped with 70% isopropanol. The heat seals were cut off from the straw, and the contents were expelled in M2 medium within a 35 mm Petri dish. The recovered embryos were rinsed two times in fresh M2 medium. The embryos were then placed into KSOM+AA culture media in the incubator until they expanded to blastocysts.

After 24 hr culture, those embryos that developed to the expanded blastocyst stage were plated on mitomycin C-treated mouse embryonic fibroblasts (MEF) cells obtained from CF-1 strain mice. The ES cell culture medium for this step contained Knockout DMEM (Invitrogen/GIBCO, catalog no. 11885084) supplemented with 15% knockout serum replacement (KSR; Invitrogen/GIBICO, catalog no10828-028), 1,000 units/ml leukemia inhibitory factor (LIF; Chimicon, Temecula Calif., catalog no. ESG1107), 0.1 mM 2-mercaptophenol (Sigma), 2 mM glutamax (Invitrogen), 1 mM sodium pyruvate, and 0.1 mM nonessential amino acids.

After 5 days of cultivation, the cultures were inspected for any inner cell mass (ICM) outgrowth and the ICM outgrowths that had formed were individually disaggregated. For this, the ICM outgrowth was picked up with a micropipette and disaggregated into single cells with 0.5% trypsin-EDTA. The disaggregated cells were seeded onto a fresh feeder layer of MEFs in 4-well plates. Cells were then further split and passaged according to methods known to those of skill in the art. Subculturing was performed at intervals of 2-3 days between splits after passage 3. The cultured embryos and ICM outgrowths were monitored for morphology after 48 hrs, 72 hrs, 96 hrs, and 120-hr after thawing. Normal morphology was observed at all these time points. The subcultured cells also exhibited normal morphologies both on feeder cells an without feeder cells.

The thawed C57BL/6J embryos were cultured on MEF feeders to establish ES cell lines. Six ES cell lines were established from cryopreserved embryos after freezing. The rate of ES cell lines generation was high in cryopreserved embryos (Table 1). The ES cell cultures obtained were tested for their developmental capacity by production of chimeras and assessment of ES cell contribution to the germline of the chimeras generated. Two C57BL/6 ES cell cultures, MK6 and MK6V, exhibited high germline contribution in chimeric mice, indicating a high developmental capacity. Two ES cell lines, designated MK6 and MK6V, were established from these cultures.

TABLE 1
Derivation of ES cell lines from cryopreserved C57BL/6J embryos

| | | | ICM | Cultures | ES cell |
|---|---|---|---|---|---|
| Genetic | | Embryos | out- | w/ES-like | lines es- |
| background | Embryos | cultured | growths | colonies | tablished |
| C57BL/6J | Frozen | 16 | 11 (69%) | 7 (44%) | 6 (37.5%) |

### Example 4

### Gender of the Established ES Cell Lines

The gender of the established ES cell lines was determined by PCR with the following primers that amplify a section of the mouse sry1 gene.

Sense Primer:
(SEQ ID NO: 1)
AACAACTGGGCTTTGCACATTG
Antisense Primer:
(SEQ ID NO: 2)
GTTTATCAGGGTTTCTTCTAG

In a PCR assay using these primers, male C57BL/6 cells (XY) will generate two bands-166 bp (Y) and 146 bp (X), while female cells (XX) will have only the 146 bp (X) band. The two C57BL/6J ES cell lines MK6 and MK6V both generated a 166 bp and a 146 bp band, indicating that they are both male cell lines.

### Example 5

### Chromosome Number Analysis

For chromosomal analysis, ES cells were arrested in metaphase by treatment with colcemid at a final concentration of 0.02 μg/ml for 1 hour. The ES cells were then trypsinized, swelled with 75 mM KCl hypotonic solution, fixed with a solution of methanol and acetic acid (3:1), and prepared on slides. Chromosomes were stained with 5% Giemsa solution. The chromosome numbers of 50 well spread metaphases for each ES cell line were evaluated by Nikon 50i microscopy. Both C57BL/6J ES cell lines, MK6 and MK6V, showed a normal euploid chromosomes number in cells obtained from passage number 15.

### Example 6

### In Vivo Differentiation

To induce tumor formation in nude mice, about 200 cells of the MK6 and MK6V ES cell lines from confluent plates were injected into the rear leg muscles of 4 weeks-old mice. The nude mice were killed 4-6 weeks after ES cell injection and the resulting teratomas were fixed with 10% buffered formalin, decalcified, embedded in paraffin and sectioned.

The histological analysis revealed that tumors from both cell lines contained differentiated cells of all three germ layers, including squamous epithelium (ectoderm), mesoderm muscle cells cartilage-like structure and bone (mesoderm), and colonic gut epithelium (endoderm), which is consistent with the high developmental capacity of the ES cell lines.

### Example 7

### Generation of Chimeric Mice by Injection into Albino Albino-C57BL/6J cBrd/cBrd/cr and Balb/c Blastocysts

To further evaluate the functionality of the newly established inbred C57BL/6 ES cell lines MK6 and MK6V, their performance was tested by diploid blastocyst injection. To generate chimeric mice, cells of the MK6V or MK6 ES cell lines were injected into albino-C57BL/6J cBrd/cBrd/cr or BALB/c host blastocysts. The resulting offspring was chimeric as judged from contribution to the coat color. Further, for assessment of ES cell germline contribution, the chimeras were bred and were found to pass the ES cell genome on to their offspring, as judged from offspring exhibiting the black coat color characteristic for the C57BL/6J strain after mating with non-black females. The observed chimerism observed by coat color was about 90-100% for BL/6 albino host blastocysts, and about 70-80% for Balb/c host blastocysts.

The results described in Table 2 demonstrate that cells from both the MK6 and MK6V ES cell lines produce high contribution chimeras and contribute to the germline of chimeras at a high frequency when injected into B6-albino and Balb/c host blastocysts.

MK6 cells contributed to 74% (14/19) of the chimeras produced by ES cell injection into albino-B6 host blastocysts and generated 55.5% (5/9) germline transmission. In contrast, MK6V ES cells contributed to 100% (21/21) of the chimeras produced and exhibited 80% germline transmission. The MK6V line further generated full ES-cell derived 100% black coat color chimeras, indicating that the injected ES cell outcompeted the host embryo's ICM cell during development, which is consistent with a higher developmental capacity than the ICM cells of the host blastocyst. Nine out of 21 (41%) of the chimeras generated from diploid embryos injected with MK6V cells were 100% ES cell-derived chimeras as judged by coat color.

Some of the pups derived from these experiments showed 100% contribution of the injected ES cell lines as judged by coat color and produced only black offspring in subsequent matings, suggesting that both the coat and the germline were essentially completely derived from the injected C57BL/6 ES cells.

TABLE 2
Generation of germline chimeric mice from diploid blastocyst injected with C57BL/6 ES cells

| | | | No. of | | | | |
|---|---|---|---|---|---|---|---|
| | Cell | Host | embryos | No. of | | Male | Germline/ |
| Experiment | lines | embryos | transferred | pups born | Chimeras | chimeras | tested |
| 1 | MK6 | B6-albino | 40 | 19 (48%) | 14 (74%) | 11 (79%) | 5/9 (56%) |
| 2 | MK6 | BALB/c | 27 | 23 (85%) | 10 (43%) | 9 (90%) | 3/5 (60%) |
| 3 | MK6V | B6-albino | 44 | 21 (48%) | 21 (100%) | 21 (100%)* | 4/5 (80%) |

B6-albino (C57BL/6J/cBrd/cBrd/Cr), Black coat color of the resulting offspring indicated germline transmission.
*9 males were 100% black coat color chimeras

### Example 8

### Tetraploid Complementation Experiments

B6D2F1 females (3 to 4 weeks old) were superovulated by injections of 5 IU of pregnant mare's serum (PMS) followed 48 hrs late with 5 IU human chorionic gonadotropin (hCG). After administration of hCG, females were mated with B6D2F1 males Fertilized zygotes were collected from the mice and incubated overnight at 37° C., 5% CO₂ in KSOM plus amino acids (KSOM+AA) media to obtain 2-cell embryos. The blastomeres of the 2-cell diploid embryos were electrofused individually to produce one-cell tetraploid embryos using a 30 μsec, 40 V pulse in a CF-150/B electrofuser (Biological Laboratory Equipments Hungary. Electrofusion was carried out in a 0.3M mannitol solution.

More than 95% percent of diploid 2-cell stages were successfully fused to produced 1-cell tetraploid embryos (fusion blastomeres) in 30-50 minutes. The tetraploid embryos were then incubated in KSOM+AA media for 48 hours to produce tetraploid blastocysts. For tetraploid blastocyst complementation, 10-15 ES cells, either MK6 or MK6V, were injected into the blastocoele of each tetraploid blastocyst. Complemented blastocysts were then cultured in KSOM+AA media for 3-4 hrs before transfer into pseudopregnant CD-1 female recipients (2.5 days post coitum; Charles River Laboratories). For each pseudopregnant CD-1 recipient, 9-11 re-expanded blastocysts were transferred into each uterus horn.

### Example 9

### Generation of Completely ES Cell-Derived Mice by Injection of Tetraploid Blastocysts

As shown in Table 3, tetraploid blastocysts complemented with either MK6 or MK6V ES cells generated live pups at birth at an average of 19%, and an average of 63% of live pups at birth survived to adulthood. Mice generated after tetraploid embryo complementation were fertile. Both ES cell lines exhibited a similar average survival rate after birth of a live pup with 64% and 62% for MK6V and MK6, respectively (Table. 3)

TABLE 3
Generation of completely ES cell-derived mice from tetraploid
blastocyst injected with C57BL/6 ES cells

| | | No. of | | | |
|---|---|---|---|---|---|
| | | embryos | No. of | No. of | Adult |
| Experiment | Cell lines | transferred | live pups | surviving | ES mice |
| 1 | MK6V-P3 | 24 | 3(13%) | 2(67%) | 2(100%) |
| 2 | MK6V-P5 | 66 | 15(23%) | 9(60%) | 9(100%) |
| 3 | MK6-P4 | 59 | 7(12%) | 4(57%) | 4(100%) |
| 4 | MK6-P6 | 60 | 9(15%) | 6(67%) | 6(100%) |
| 5 | MK6-P7 | 46 | 13(28%) | 8(62%) | 8(100%) |

### Example 10

### Generation of Genetically Modified Mice Derived from C57BL/6J ES Cells

It was investigated whether C57BL/6J ES cells can be used to generate genetically modified mice using tetraploid injections. MK6 ES cells were used for this set of experiments to generate knockout mice by targeted recombination. Specifically, the line was assessed to show 86% euploid cells (40 chromosomes) at passage 5, and showed typical ES colony morphology. Targeted disruption of 2 genes of interest in MK6 cells was accomplished by homologous recombination. For this, ES cells were electroporated (Gene Pulser, BioRad, Hercules, Calif.) with linearized targeting vector containing a neomycin resistance gene. The cells were subsequently cultured in the presence of G418 for 8 days. The G418-resistant ES colonies were picked and cultured in 96-well plates for 3 more days. Duplicated 96-well ES cell plates were screened and positive colonies were genotyped by PCR. Cells from two correctly targeted ES cell clones, F9 and C4, where injected into tetraploid blastocysts in order to generate of genetically modified mice. This tetraploid complementation experiment resulted in an average of 18% live pups at birth, of which an average of 43% survived to adulthood (Table.4). Accordingly, the ES cell derivatives developed in this experiment (here: gene-targeted ES cell subclones) exhibited a developmental capacity similar to the parent ES cell line in the tetraploid complementation assay.

The targeting vectors were designed to insert an H2B-GFP and SV40 ployA signal, and an Frt-Flanked Neo cassette was cloned into the initiation codon of the H2BGFP fusion gene. The targeting vector AI6 was obtained from the Allen Institute. PCR Primers (5′ atttagct-3′) were used to amplify the SA fragment using the plasmid pMB80 (R26-creER) as a template. The fragment was cloned into the pROSA26-1 vector to obtain the pROSA26-SA-construct. An Fse I restriction enzyme site was added to the primer pairs used for PCR amplification of H2B-GFP from the template DNA (DNA ID: 11680 (H2B-GFP 5107 bp) purchased from Addgene):

(SEQ ID NO: 3)
5′TTTAAAGGCCGGCCATGCCAGAGCCAGCGAAGTCTG3′
(SEQ ID NO: 4)
5′TTTAAAGGCCGGCCTTACTTGTACAGCTCGTCCATG3′

The backbone plasmid AI6 was digested with Fse I to subclone H2B-GFP and the direction of the insert was verified. The construct was confirmed to be correct by sequencing. R26-H2B-GFP was digested with KpnI and this (linear) construct was electroporated into MK6 embryonic stem cells (C57BL/6 genetic background), which were then selected for resistance to G418 in 160 ug/ml for 8 days.

Individual G418-resistant colonies were picked into 96-well plated and isolated. ES cells in which the floxed cassette and H2B sequences were correctly inserted and exclusively present in the Rosa26 locus were identified by PCR and Southern blots with 5′-arm and 3′-arm as probes. Correctly targeted ES cells (F9, C4) were then isolated and injected into recipient tetrapolid blastocysts, which were transferred into the uterus of a foster CD-1 mother. Eighteen days later, pups were born and fully ES cell-derived mouse founders were identified by coat color.

PCR Primers for pRosa26 were used for PCR-genotyping of mice to distinguish WT, heterozygous, and homozygous mice:

(SEQ ID NO: 5)
(Rosa1): CCCAAAGTCGCTCTGAGTTGTTATC
(SEQ ID NO: 6)
(Rosa2): GAAGAAGCGGGAGAAATGGATATG
(SEQ ID NO: 7)
(Cag3): CCAGGCGGGCCATTTACCGTAAG

The R26-H2B-GFP mouse line was kept in the C57BL/6J genetic background

### Electroporation in MK6 ES Cells

MK6 ES cells are routinely passaged three days prior to electroplating. One 10 cm plate at approximately 80% confluency was used for the electroporations. Electroporation was carried out as follows:
1. ES medium on ES cells was changed 2 hours prior to electroporation.
2. ES cells were trypsinized and ES cell culture medium was added to inhibit the action of the trypsin.
3. The cells were pipetted up and down to produce a single-cell suspension at a densitiy of 1×10⁷ cells/ml in PBS.
4. For electroporation, 0.8 ml of the cell suspension was placed into a cuvette (Bio-Rad Cat#165-2088).
5. 20 μg of linear DNA was added to the cells and mixed. The mixture was incubated at room temperature for 5 minutes.
6. A single pulse of 250V, 500 μF was applied to the cells using a Bio-Rad Gene Pulser with a Capacitance Extender.
7. After applying the pulse, the cells were allowed to stand at room temperature for 5 minutes.
8. The cells were then removed from the cuvette and diluted in an appropriate volume of ES cell medium
9. 10 ml of the resuspended ES cells were transferred onto each of a number of 10-cm tissue culture dishes of feeder cells
10. The ES cell medium was changed on the next morning and G418-drug selection was begun (160 ug/ml).
11. After 8 days of selection, individual drug-resistant colonies appeared and were large enough to pick up.
12. For colony picking, sets of 96-well tissue culture plates were prepared to contain G418-resistant MEF feeder cells. 96 (1×96 well plate) colonies were picked, one into each well of a picking plate, and the plate was split into two duplicates. One plate was used for cryopreservation of the ES cell colonies in an −80° C. freezer. The other plate was used for DNA screening.

The gene-targeted ES cell subclones F9 and C4 yielded 16% and 20% of fully ES cell-derived mice, respectively. All mice generated in this experiment were male with completely black coat. The efficiency of tetraploid complementation, both in terms of percentage of live pups in relation to complemented tetraploid embryos transferred and in terms of surviving adult mice was significantly higher than that reported in previous publications for inbred C57BL/6 ES cell lines, for example, in a report using inbred C57BL/6 ES cells aggregated with tetraploid blastomeres (Eggan et al., PNAS 2001; Li et al., Reproduction 2005; and Tanimoto Y et al, 2008). The results are summarized in Table 4.

TABLE 4
Generation of completely ES-cell derived mice from tetraploid
blastocyst Injected with gene-targeted ES clones

| No. of | Injected | No. of | | | Adult |
|---|---|---|---|---|---|
| Positive | ES cell | embryos | No. of | No. of | ES-cell |
| clones | clones | transferred | live pups | surviving | mice |
| 13/96 | F9 | 50 | 10(20%) | 6(60%) | 4(67%) |
| (13.5%) | | | | | |
| | C4 | 37 | 6(16%) | 4(67%) | 3(50%) |

The direct production of genetically modified mice on an C57BL/6 background without the need to backcross has been difficult. The host blastocyst donor for diploid blastocyst injections is commonly a BALB/c inbred mouse. However, it is difficult to get sufficient numbers of high-quality blastocysts from BALB/c mice. Another useful mouse strain used for host blastocyst generation is the co-isogenic, albino C57BL/6J TyrC2J (Jackson laboratory), which gives rise to mice with white coat color. However, this albino white C57BL/6J strain is expensive and typically not commercially available in sufficient numbers needed for blastocyst injection experiments. Further, most facilities generate genetic modifications in ES cells of the 129 strain and use C57BL/6 donor blastocysts for the production of chimeras.

The data described here demonstrate that the ES cells, ES cell lines, derivatives, and methods provided herein can improve the efficiency of generating genetically modified mice in a C57BL/6J background. C57BL/6J-cBrd/cBrd/cr (NCI) host embryos performed best in combination with the C57BL/6J inbred ES cell lines provided herein to generate genetically modified C57BL/6 mice.

### Example 11

### Characterization of Spermatozoa of ES Cell Derived Mice

To characterize the spermatozoa of C57BL/6 ES cell derived chimeras and pups generated after tetraploid blastocyst complementation, testes and caudae epidiymidis were excised from these mice and sperm cells were collected in HTF medium with BSA. The sperm cells were counted and the motility of the sperm cells was analyzed with a Cell-Vu sperm counting chamber. No differences in total sperm concentration and motility were detected between inbred C57BL/6 mice, C57BL/6 inbred ES cell chimeras, and C57BL/6 inbred ES cell derived pups generated by tetraploid blastocyst complementation. Testes were also sectioned and no morphological or pathological abnormalities were observed in the ES cell derived chimeras or mice generated from tetraploid blastocyst complementation.

### REFERENCES

Altman, P L and D D Katz, 1979. Inbred and genetically defined strains of laboratory animals Part I: Mouse and rat. Federation of American Societies for Experimental Biology.
Austin, C P, Battery J F, Bradley A, Bucan M, Capecchi M, Collins F S, Dove W F, Duyk G, Dymecki S, Eppig J T et al. 2004. The knockout mouse project. Nat. Genet. 36: 921-924
Doetschman T, Gregg R G, Maeda N, Hooper M L, Melton D W, Thompson S, Smithies O. 1987. Targeted correction of a mutant HPRT gene in mouse ES cells. Nature 330:576-578.
Evans M J, Kaufman M H. 1981. Establishment in culture of pluripotential cells from mouse embryos. Nature 292:154-156
Joyner A. L. (2000). Gene Targeting A Practical Approach 2^{nd} edn. Oxford Uni. Press. P. 189-192. Production and analysis of ES cell aggregation chimeras. (A. Nagy and J. Rossant)
Leibo S P. 1986, Genetic Engineering of Animals (J W Evans and A Hollaender Eds. Plenum Press, pp 251-272)
Martin G R. 1981. Isolation of a pluripotent cell line from early mouse embryos cultured in medium conditioned by teratocarcinoma stem cells. Proc. Natl. Acad Sci USA 78:7634-7638
Kontgen F, Suss G, Stewart C, Steinmetz M, Bluethmann 1993. Int. Immunol. 5:957-964
Pettitt S J, Liang Q, Rairdan X Y, Moran J L, Prosser H M, Beier D R, Lioyd K C, Bradlru A, Skarnes W C. 2009. Agouti C57BL/6N ES cells for mouse genetic resources. Nature method. 6:493-495
Tanimoto Y, Iijima S, Hasegawa Y, Suzuki Y, Daitoku Y, Mizuno S, Ishige T, Kudo T, Takahashi S, Kunita S, Sugiyama F, and Yagami K I. 2008. ES cells derived from C57BL/6J and C57BL/6N mice. Comparative medicine. 58:347-352

All publications, patents and sequence database entries mentioned herein, including those items listed below, are hereby incorporated by reference in their entirety as if each individual publication or patent was specifically and individually indicated to be incorporated by reference. In case of conflict, the present application, including any definitions herein, will control.

### EQUIVALENTS AND SCOPE

Those skilled in the art will recognize, or be able to ascertain using no more than routine experimentation, many equivalents to the specific embodiments of the invention described herein. The scope of the present invention is not intended to be limited to the above description, but rather is as set forth in the appended claims.

It should be understood that, where a mouse strain is provided and the description of an embodiment or aspects of the invention, any specific substrain known to those of skill in the art can be excluded from the embodiment. For example, an embodiment including ES cells derived from inbred C57BL/6 embryos can include ES cells derived from any or from a specific C57BL/6 substrain, for example, from the C57BL/6J substrain and/or the C57BL6/N substrain. For the sake of brevity, not all iterations and combinations of substrains are spelled out for each embodiment.

What is claimed is:

1. An inbred mouse C57BL/6 embryonic stem (ES) cell that exhibits high developmental capacity.

2. The inbred mouse C57BL/6 ES cell line of claim 1, wherein the ES cell line is a C57BL/6J ES cell line.

3. The inbred mouse ES cell of claim 1, which exhibits substantially the same developmental capacity as inbred ES cell line MK6 or substantially the same developmental capacity as inbred ES cell line MK6V.

4. The inbred mouse ES cell of claim 3, wherein the ES cell is derived from the MK6 ES cell line or the MK6V ES cell line.

5. A mouse ES cell population, comprising
one or more inbred mouse C57BL/6 ES cells that exhibit high developmental capacity; or
one or more inbred mouse C57BL/6J ES cells that exhibit substantially the same developmental capacity as inbred ES cell line MK6 or substantially the same developmental capacity as inbred ES cell line MK6V, or that are derived from the MK6 ES cell line or the MK6V ES cell line.

6. The mouse ES cell population of claim 5, wherein the ES cell population contributes to the germline in pups generated after tetraploid embryo complementation.

7. The mouse ES cell population of claim 5, comprising at least one cell that comprises a genetic modification.

8. The mouse ES cell population of claim 7, wherein essentially all cells in the population comprise the same genetic modification.

9. The mouse ES cell population of claim 8, wherein the genetic modification is a heterologous nucleic acid construct stably integrated into the genome of the cell.

10. The mouse ES cell population of claim 8, wherein the genetic modification is a knockout, a knock-in, a viral vector, or a randomly integrated nucleic acid construct.

11. A pre-implantation embryo comprising
one or more inbred mouse C57BL/6 ES cells that exhibit high developmental capacity; or
one or more inbred mouse C57BL/6J ES cells that exhibit substantially the same developmental capacity as inbred ES cell line MK6 or substantially the same developmental capacity as inbred ES cell line MK6V, or that are derived from the MK6 ES cell line or the MK6V ES cell line.

12. The pre-implantation embryo of claim 11, wherein the pre-implantation embryo is a blastocyst.

13. The pre-implantation embryo of claim 11, wherein the embryo comprises a tetraploid trophoblast cell population.

14. The pre-implantation embryo of claim 11, wherein the cell population of the embryo consists of the ES cell population of the one or more ES cells and a tetraploid cell population.

15. A mouse comprising a cell derived from
an inbred mouse C57BL/6 ES cell that exhibits high developmental capacity; or
an inbred mouse C57BL/6J ES cell that exhibits substantially the same developmental capacity as inbred ES cell line MK6 or substantially the same developmental capacity as inbred ES cell line MK6V, or that is derived from the MK6 ES cell line or the MK6V ES cell line.

16. The mouse of claim 15, wherein the mouse consist essentially of cells derived from
one or more inbred mouse C57BL/6 ES cells that exhibit high developmental capacity; or
one or more inbred mouse C57BL/6J ES cell that exhibit substantially the same developmental capacity as inbred ES cell line MK6 or substantially the same developmental capacity as inbred ES cell line MK6V, or that are derived from the MK6 ES cell line or the MK6V ES cell line.

17. A differentiated cell derived from
an inbred mouse C57BL/6 ES cell that exhibits high developmental capacity; or
an inbred mouse C57BL/6J ES cell that exhibits substantially the same developmental capacity as inbred ES cell line MK6 or substantially the same developmental capacity as inbred ES cell line MK6V, or that is derived from the MK6 ES cell line or the MK6V ES cell line.

18. A mouse ES cell of high developmental capacity, wherein the ES cell is derived from an inbred C57BL/6 embryo, and wherein the ES cell, or pluripotent cells derived from the ES cell or substantially identical to the ES cell, generate live pups after tetraploid blastocyst complementation.

19. The mouse ES cell of claim 18, wherein the ES cell, or pluripotent cells derived from the ES cell or substantially identical to the ES cell, generate more than 5%, more than 10%, more than 15%, more than 20%, or more than 25% live pups (live pups at term/tetraploid complemented embryos transferred) after tetraploid blastocyst complementation.

20. The mouse ES cell of claim 18, wherein the tetraploid blastocysts are cBrd/cBrd/cr or BALB/c blastocysts.

21. The mouse ES cell of claim 18, wherein the embryo is a C57BL/6J embryo.

22. The mouse ES cell of claim 18, wherein the embryo has been frozen.

23. The mouse ES cell of claim 18, wherein the embryo has been cryopreserved.

24. The mouse ES cell of claim 23, wherein the embryo has been cryopreserved at the eight-cell stage.

25. The mouse ES cell of claim 23, wherein the embryo has been cryopreserved for at least 3 months, at least 6 months, at least 1 year, at least 2 years, or at least 3 years.

26. The mouse ES cell of claim 18, wherein the cell exhibits a normal karyotype.

27. The mouse ES cell of claim 18, wherein the cell exhibits a 40,XY karyotype.

28. The mouse ES cell of claim 27, wherein the pups generated are male.

29. The mouse ES cell of claim 18, wherein the cell exhibits a 39,X0 karyotype.

30. The mouse ES cell of claim 29, wherein the pups generated are female.

31. A cell derived from the ES cell of claim 18, wherein the cell is a pluripotent cell or a differentiated cell.

32. The cell of claim 31, wherein the cell is comprised in a mouse.

33. The cell of claim 31, wherein the cell comprises a genomic modification.

34. The cell of claim 31, wherein the cell comprises a heterologous nucleic acid construct stably inserted into its genome.

35. A mouse comprising the cell of claim 31.

36. A mouse, wherein the mouse is generated by injection of at least one pluripotent cell derived from
one or more inbred mouse C57BL/6 ES cells that exhibit high developmental capacity; or
one or more inbred mouse C57BL/6J ES cells that exhibit substantially the same developmental capacity as inbred ES cell line MK6 or substantially the same developmental capacity as inbred ES cell line MK6V, or that are derived from the MK6 ES cell line or the MK6V ES cell line;
into a host blastocyst.

37. The mouse of claim 36, wherein the blastocyst is a diploid blastocyst.

38. The mouse of claim 36, wherein the blastocyst is a tetraploid blastocyst.

39. The mouse of claim 38, wherein essentially all cells of the mouse are derived from the injected one or more ES cells.

40. The mouse of claim 36, wherein at least one germ cell of the mouse is derived from the injected one or more ES cells.

41. A method for deriving a mouse ES cell line from a frozen or cryopreserved embryo, the method comprising
(a) providing a frozen or cryopreserved, inbred C57BL/6 mouse embryo;
(b) culturing the embryo until the blastocyst stage;
(c) incubating the blastocyst on feeder cells for a time sufficient and under conditions appropriate to form an inner cell mass (ICM) outgrowth;
(d) disaggregating the inner cell mass outgrowth; and
(e) subculturing an ES cell population derived from the ICM outgrowth to establish an ES cell line.

42. The method of claim 41, further comprising thawing the embryo.

43. The method of claim 42, wherein
the step of (b) comprises culturing the embryo in KSOM-AA medium; and/or
the step of (c) comprises incubating the blastocyst on feeder cells in ES cell culture medium.

44. The method of claim 41, wherein the embryo has been frozen or cryopreserved at the eight-cell stage.

45. The method of claim 41, wherein the embryo has been frozen or cryopreserved for at least 3 months, at least 6 months, at least 1 year, at least 2 years, or at least 3 years.

46. The method of claim 43, wherein the ES cell culture medium comprises,
(i) Knockout DMEM (Invitrogen/GIBCO);
(ii) knockout serum replacement (KSR, Invitrogen/GIBCO);
(iii) 1,000 units/ml leukemia inhibitory factor (Chimicon);
(iv) 0.1 mM 2-mercaptophenol (Sigma);
(v) 2 mM glutamax (Invitrogen);
(vi) 1 mM sodium pyruvate; and/or
(vii) 0.1 mM nonessential amino acids.

47. An embryonic stem cell, wherein the ES cell is derived from a frozen or cryopreserved inbred C57BL/6 embryo, and wherein the ES cell is able to proliferate in an undifferentiated state for more than 1 year when cultured in the presence of LIF on feeder cells, and wherein the ES cell is able to generate live pups at a frequency of at least 5%, at least 10%, at least 15%, or at least 20% in a tetraploid embryo complementation assay.

48. The embryonic stem cell of claim 47, wherein the embryo is a C57BL/6J embryo.
